(19) United States
(12) Patent Application Publication
(10) Pub. No.: US 2026/0076750 A1
(43) Pub. Date: Mar. 19, 2026

(54) Enhancing Awake Neurosurgery With A Patient-Worn Head-Mounted Device

(71) Applicant: Stryker European Operations Limited, Carrigtwohill (IE)

(72) Inventor: Florian Herrmann, Schwanau (DE)

(73) Assignee: Stryker European Operations Limited, Carrigtwohill (IE)

(21) Appl. No.: 19/327,323

(22) Filed: Sep. 12, 2025

Related U.S. Application Data
(60) Provisional application No. 63/694,266, filed on Sep. 13, 2024.

(51) Int. Cl. A61B 34/20; A61B 5/00; A61B 5/11; G06F 3/01; G06T 7/70; G06V 20/50; H04R 1/02

(52) U.S. Cl. CPC A61B 34/20; A61B 5/1103; A61B 5/1104; A61B 5/4064; A61B 5/7405; A61B 5/7445; A61B 5/746; G06F 3/013; G06F 3/017; G06T 7/70; G06V 20/50; G06T 2207/30016; G06V 2201/031; G06V 2201/034; H04R 1/028

(57) ABSTRACT

Techniques for enhancing awake brain surgery include a navigation system to track a pose of a surgical instrument relative to a pose of the target anatomy of a patient. A head-mounted device (HMD) is worn by the patient during surgery. A control system is in communication with the navigation system and the HMD. The control system registers one or more images with the target anatomy. The control system identifies a structure of the target anatomy based on the one or more images of the target anatomy. The control system detects the pose of the surgical instrument being proximate to the identified structure of target anatomy, and based on such, the control system controls the HMD to generate an output to provoke a response of the patient.

### CROSS REFERENCE TO RELATED APPLICATIONS

The subject application claims priority to and all benefits of U.S. Provisional Patent App. No. 63/694,266, filed Sep. 13, 2024, the entire contents of which are hereby incorporated by reference.

### BACKGROUND

In awake brain surgery or awake craniotomy, the patient remains conscious during a portion of the surgery. Such open craniotomy surgeries require a portion of the skull to be removed to access the brain to remove tumors, blood clots, and/or other diseased tissues. To remove diseased tissues, a surgeon will need to access the brain without damaging brain tissue that is healthy and/or control functions of the body.

Patients undergoing awake brain surgery face several challenges. Remaining awake during brain surgery can be emotionally distressing for many patients. The fear and anxiety associated with the procedure can be overwhelming. Patients may experience pain and discomfort during the surgery, e.g., because of scalp incisions, skull drilling, and exposure of the brain. Local anesthesia is used to minimize pain, but patients may still feel pressure and discomfort. Communication is essential during awake brain surgery to ensure that critical brain functions like speech, movement, and sensation are preserved. However, patients may find it challenging to interact with the surgical team while staying still and conscious. Awake brain surgery often involves tasks that test cognitive functions, such as language and memory. Patients may struggle to perform these tasks due to anxiety and the presence of surgical instruments in their brain. Additionally, the positioning of the patient during surgery and draping of the sterile field leaves the patient in an uncomfortable situation, while neurological tests require the patient to focus and concentrate. Furthermore, awake brain surgery can be time-consuming since the surgeon typically needs to take breaks to assess the patient's neurological function. This can extend the duration of the procedure and increase patient discomfort.

To determine which tissue areas can and cannot be removed by the surgeon, baseline responses directed towards a patient's physical and mental capabilities must be determined during surgery so that the surgical team can evaluate whether tissue removal will cause permanent damage to the patient's brain.

To establish the baseline responses corresponding to the patient's mental and physical capabilities, a litany of questions and commands may be given to the patient to establish their baseline responses (e.g., “normal” mental and physical state). Traditional methods of evaluating the patient's mental and physical abilities may be one or more members of the medical team asking questions and/or giving commands and observing the patient to establish the patient's baseline responses.

Although some instrumentation may be implemented to assist in the evaluation of the patient's functions, there is a need for a better and more precise implementation and data collection system. There is also a need for continued monitoring and processing of patient information pre-operatively, intraoperatively, and post-operatively, which can be stored and presented to the medical team.

### SUMMARY

This Summary introduces a selection of concepts in a simplified form that are further described throughout the present application. This Summary is not intended to limit the scope of the claimed subject matter nor identify key features or essential features of the claimed subject matter.

According to a first aspect, a surgical system is provided, comprising: a navigation system configured to track a pose of a surgical instrument relative to a pose of a target anatomy of a patient; a head-mounted device (HMD) configured to be worn by the patient during surgery; a control system in communication with the navigation system and the HMD, the control system configured to: detect the pose of the surgical instrument being proximate to a specific structure of target anatomy; and in response, control the HMD to generate an output to the patient.

According to a second aspect, a surgical system is provided, comprising: a navigation system configured to track a pose of a surgical instrument relative to a pose of a target anatomy of a patient; a head-mounted device (HMD) configured to be worn by the patient during surgery; and a control system in communication with the navigation system and the HMD, the control system configured to: register one or more images with the target anatomy; identify a structure of the target anatomy based on the one or more images of the target anatomy; detect the pose of the surgical instrument being proximate to the identified structure of target anatomy; and based on detection of the pose of the surgical instrument being proximate to the identified structure, control the HMD to generate an output to provoke a response of the patient.

According to a third aspect, a head-mounted device (HMD) is provided for use with a navigation in awake brain surgery of a patient, the HMD comprising: a support structure to be worn by the patient during surgery; a display supported by the support structure and being visible by the patient; a speaker system supported by the support structure and being audible to the patient; one or more sensors supported by the support structure; and an HMD controller coupled to the display, speaker system, and one or more sensors, wherein the HMD controller is configured to: obtain an instruction from a navigation system in response to the navigation system detecting that a surgical instrument is proximate to a specific brain region of the patient; based on receipt of the instruction, control one or both of the display and the speaker system to generate an output to provoke a response of the patient; and utilize the one or more sensors to monitor the response of the patient and functionally evaluate the specific brain region.

According to a fourth aspect, a surgical system is provided, comprising a navigation system configured to track an instrument and a target anatomy of a patient, a head-mounted display worn by the patient, the head-mounted display including a sensor configured to monitor the patient, and a control system in communication with the navigation system and the head-mounted display. The control system is configured to determine a position of the probe and the target anatomy, provide electrical stimulation from a probe to the target anatomy of the patient, identify the response from the patient with the head-mounted display when an electrical stimulation is provided to the target anatomy by the probe based on the response of the patient, evaluate a response of the patient with the sensor of the head-mounted display based on the position of the probe and the target anatomy, and provide an indication of the evaluation with an indicator of the system.

According to a fifth aspect, the present teachings provide a smart surgical system comprising a surgical instrument, a navigation system, a head-mounted display for determining at least one parameter of a patient, the head-mounted display including a first sensor configured to monitor the at least one parameter of the patient, an alert module, and a control system. The control system is configured to receive data from the navigation system and the head-mounted display, determine a position of the surgical instrument, determine a position of a target anatomy of the patient, identify a first state of the patient based on one of an output from the first sensor and an output of the navigation system, and control the alert module based on the position of the surgical instrument, position of the target anatomy, and the first state of the patient.

A method of operating any of the above aspects is provided. An HMD for use with any of the above aspects is provided.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a perspective view of a surgical system, according to one implementation.

FIG. 2 is a schematic view of an example control system including an extended reality head-mounted display that may be used with the surgical system.

FIG. 3 is a schematic view of a portion of the surgical system including the extended reality head-mounted display.

FIG. 4A illustrates a top-down brain with annotated critical brain regions.

FIG. 4B illustrates a perspective view of the left side of the brain annotated with critical brain regions.

FIG. 5 is one example of patient images for use with the surgical system.

FIG. 6 is an alternative patient image for use with the surgical system.

FIGS. 7A-7D illustrate an example of a portion of a surgical procedure involving mapping the brain with a surgical tool relative to target anatomy and critical brain regions.

FIGS. 8A-8D illustrate an example of a portion of a surgical procedure of removing the target anatomy with a surgical tool.

FIG. 9 is an example flow diagram depicting a surgical method during a procedure utilizing extended reality with a head-mounted display worn by the patient.

FIG. 10 is an example flow diagram of a surgical method of brain mapping utilizing a head-mounted display worn by the patient.

### DETAILED DESCRIPTION

### I. Example System Overview

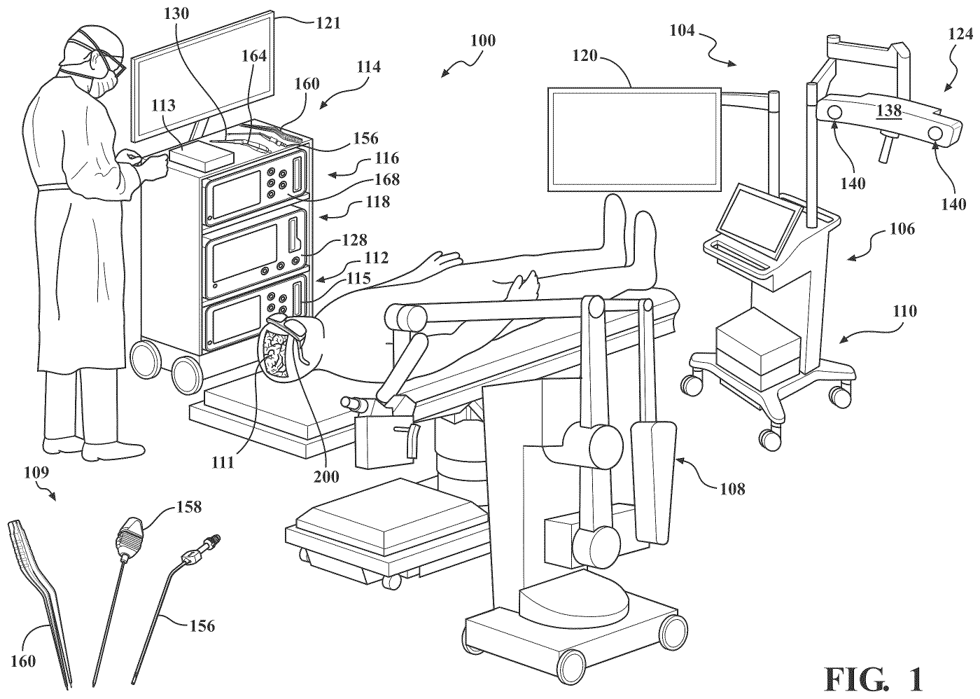

Referring to FIG. 1, a system 100 is provided. The system may be a surgical system 100 adapted for treating a patient. The surgical system 100 is shown in a surgical setting such as an operating room of a medical facility. The surgical system 100 may be used to perform any intraoperative surgical procedure on a patient. Example surgical procedures include, but are not limited to: partial knee arthroplasty, total knee arthroplasty, total hip arthroplasty, shoulder arthroplasty, spinal procedures, ankle procedures, endoscopic procedures, cranial procedures, lesion removal procedures, arthroscopic procedures, arthroscopic resection procedures, soft tissue or ligament repair procedures, neurological procedures, ENT procedures, minimally invasive MIS procedures, or the like.

In the example shown in FIG. 1, the patient is undergoing a cranial procedure. In addition, the following implementations describe the use of the surgical system 100 in performing a procedure in which material is identified and/or removed from the brain of a patient. However, it should be recognized that the surgical system 100 may be used to perform any suitable procedure in which material is removed from any suitable portion of a patient's anatomy, material is added to any suitable portion of the patient's anatomy (e.g., an implant, graft, etc.), and/or in which any other control of and/or visualization of a surgical tool is desired.

The surgical system 100 may be used to perform craniotomy procedures on a patient. During a craniotomy procedure, the patient may be awake in order to evaluate anatomical functionality (e.g., motor control, touch, smell, sight, hearing, etc.) and/or cognitive ability (e.g., following instructions, problem solving). To facilitate evaluation of a patient during a craniotomy, a baseline of the patient's motor skills and cognitive abilities are obtained pre-operatively and/or intraoperatively (e.g., while using a probe to stimulate a portion of the brain). Further, as the procedure progresses to remove tissue, evaluation tests may be administered to elicit a response from the patient in order to compare a patient's response during resection to the pre-operative and/or intraoperative evaluation results. One way to improve the evaluation process is to utilize a head mounted display 200 (HMD) on the patient. The HMD 200 includes a suite of sensors which are capable of monitoring patient outputs and recording the patient outputs in response to the prompts of the HMD 200, which will be described in detail below.

The system 100 may include a surgical navigation system 104, a surgical microscope 108, a surgical cart 114, and a suction system 113. The surgical navigation system 104 may include a cart assembly 106 that houses a navigation computer 110. The navigation computer 110 may also be referred to as the navigation controller. A navigation interface is in operative communication with the navigation computer 110. The navigation interface may include one or more input devices may be used to input information into the navigation computer 110 or otherwise to select/control certain aspects of the navigation computer 110. The navigation interface includes one or more displays 120. Such input devices may include interactive touchscreen displays/menus, a keyboard, a mouse, a microphone (voice-activation), gesture control devices, and the like.

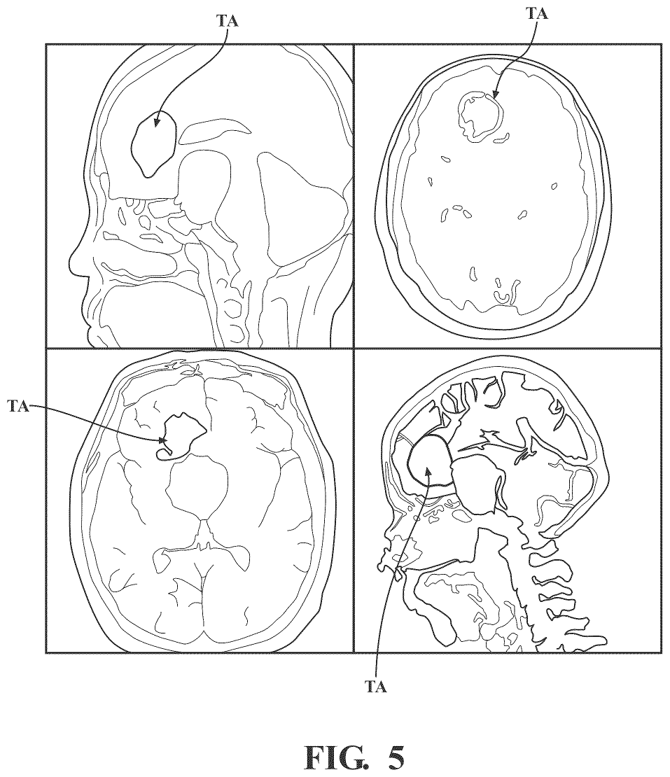

The navigation computer 110 may be configured to store one or more pre-operative or intra-operative images of the brain. Any suitable imaging device may be used to provide the pre-operative or intra-operative images of the brain. For example, any 2D, 3D or 4D imaging device, such as isocentric fluoroscopy, bi-plane fluoroscopy, ultrasound, computed tomography (CT), multi-slice computed tomography (MSCT), magnetic resonance imaging (MRI), positron emission tomography (PET), optical coherence tomography (OCT), single photon emission (see FIG. 5). The images may also be obtained and displayed in two, three or four dimensions. In more advanced forms, four-dimensional surface rendering regions of the body may also be achieved by incorporating patient data or other data from an atlas or anatomical model map or from pre-operative image data captured by MRI, CT, or echocardiography modalities.

The navigation computer 110 may generate the one or more images of the brain on a display 120. The navigation computer 110 may also be connected with the surgical microscope 108. For example, the display 120 may show an image corresponding to the field of view of the surgical microscope 108. The navigation computer 110 may include more than one display, with one such display showing the field of view of the surgical microscope 108 while the other such display may show a pre-operative or intra-operative image of the brain.

The navigation system 104 includes tracking system 124. The tracking system 124 is coupled to the navigation computer 110 and is configured to sense the position of one or more tracking elements attached to a surgical tool, the patient, and/or a head-mounted display 200. The tracking system 124 may be configured to track active or passive tracking elements attached to the surgical tool or the patient. In some examples, the trackers may be infrared. In other examples, the trackers may be RF trackers. It is also contemplated that any suitable surgical tracking techniques may be used. One non-limiting example of a surgical navigation system 104 that may be used is Nav3i™ that is commercially available from Stryker. A surgical navigation system 104 may have various functions and features as described in U.S. Pat. No. 7,725,162 B2 and U.S. Pat. No. 11,369,438 B2 which are hereby incorporated by reference in their entireties.

The surgical microscope 108 includes one or more objectives configured to provide magnification in a range (e.g., from about 2 times to about 50 times). The surgical microscope 108 may have a field of view having an area of a predetermined range. The surgical microscope 108 is configured for fluorescence microscopy, for example, to detect PPIX. The surgical microscope 108 may include one or more excitation sources (e.g., an excitation source configured to emit light in the visible light spectrum, or an excitation source configured to emit light in the infrared spectrum) for illuminating the brain tissue 111 with excitation light to cause the PPIX to fluoresce. The surgical microscope 108 may also include a camera capable of detecting radiation at the fluorescent wavelengths of PPIX or ICG.

The surgical cart 114 may include a surgical tool system 112, a suction system 113, a tissue detection system 116, and an ultrasonic surgical system 118. The surgical tool system 112 may include one or more surgical tools 109. A display 121 may be coupled to the surgical cart and operatively connected to the surgical system 112, the tissue detection system 116, and/or the ultrasonic surgical system 118 to display information related to each respective system 112, 116, and 118. A healthcare professional may use the ultrasonic surgical system 118 and/or the surgical system 112 to ablate target tissue of the brain of the patient. The ultrasonic surgical system 118 may include an ultrasonic control console 128 and an ultrasonic handpiece assembly 130.

The suction system 113 may include a suction tool 156 and suction unit to control various aspects of the suction tool 156. A suction tube may connect the suction tool 156 to the suction system 113. The suction system 113 may receive suction from a vacuum source, such as a vacuum outlet of a medical facility. The suction system 113 may include one or more regulators or one or more adjustment valves for controlling the suction pressure received from the vacuum source. The suction system 113 may also include one or more containers for storing the waste collected by the suction tool 156. In an example, the suction system 113 may correspond to a wall suction unit. In another example, the suction system 113 may correspond to a portable suction unit. The suction system 113 and the suction tool 156 may have various features, as described in U.S. Pat. No. 9,066,658 B2 and U.S. Pat. Pub. No. 2018/0344993 A1 which are hereby incorporated herein by reference in its entirety.

The surgical tool system 112 may include a surgical tool 109, such as powered forceps 160, and a surgical control console 115 to control various aspects of the surgical tool. In some examples, the powered forceps 160 may be monopolar, bipolar, or both. The healthcare professional may also use the surgical tool to perform any surgical operation on the tissue, for example, to ablate the tissue or to cauterize the tissue. The bipolar forceps may have features as described in U.S. Pat. No. 8,361,070 B2 which is hereby incorporated by reference in its entirety. While the disclosure discusses and illustrates that the surgical tool may include powered forceps 160, the surgical tool system 112 and surgical tool 109 may include other tools, such as a neuro stimulator (also referred to as a probe 158), a dissector, or an ablation device (e.g., an RF ablation device and/or a laser ablation device). The surgical tool system 112 may include other surgical tools 109 such as scalpel, monopolar forceps, needle, saw, scissors, spatula, retractor, punch, or the like. For example, the surgical system and/or surgical tools may have various features as described in U.S. Pat. No. 8,267,934 B2, which is hereby incorporated by reference in its entirety. Any number of surgical systems and any number of surgical tools may be employed by the healthcare professional in performing the surgical procedure.

The tissue detection system 116 may include a control console 168 and a sample element 164. The control console 168 may provide the healthcare professional with a real-time indication via the sample element 164 when brain tissue 111 corresponds to the target tissue. The sample element 164 may also be coupled to the powered forceps 160, the suction tool 156, or other surgical tools as will be described in greater detail below. The tissue detection system 116 determines when the brain tissue 111 corresponds to target tissue based on fluorescence emitted by the target tissue caused by the fluorophore. In an example, the fluorophore may correspond to PPIX. In another example, the fluorophore may correspond to ICG. As will be discussed in greater detail below, based on the intensity and the wavelengths of the fluorescence emitted by PPIX, the tissue detection system 116 may determine that the target tissue is present.

In some examples, the surgical tool 109 may be attached to a manipulator. Such an arrangement is shown in U.S. Pat. No. 9,119,655, entitled, “Surgical Manipulator Capable of Controlling a Surgical Instrument in Multiple Modes,” the disclosure of which is hereby incorporated by reference. In one example, the manipulator has a base, a plurality of links extending from the base, and a plurality of joints (not numbered) for moving the surgical tool 109 with respect to the base. In some examples, some or all of the joints may be passive joints or active joints. The manipulator may have a serial arm or parallel arm configuration. The manipulator may be floor mounted, ceiling mounted, gantry mounted, table mounted, or patient mounted. More than one manipulator may be utilized.

The surgical system 100 is illustrated in FIGS. 1-3 as including the surgical tool 109 which may include manually operated, hand-held, and/or robotic tools. For example, the surgical tool 109 may include a hand-held motorized saw, drill, bur, probe, or other suitable tool that may be held and manually operated by a surgeon. In the nonlimiting example shown in FIG. 1, surgical tools 109 are shown as suction tool 156, probe 158, and powered forceps 160.

The surgical system 100 includes a clinical application CA. The clinical application CA may be displayed on one or more displays 120, 121 of the navigation system 104. The clinical application CA assists a surgeon or staff in performing the surgical procedure. The clinical application CA may have a plurality of different screens related to the surgical procedure. Such screens may include a pre-operative planning screen, an operating room setup screen, an anatomical registration screen, an intra-operative planning screen, an anatomical preparation screen, or a post-operative evaluation screen, and the like. The clinical application CA may present a navigation guidance region that displays one or more of the surgical objects tracked by tracking system 124, which includes a localizer 134 of the navigation system 104.

The localizer 134 communicates with the navigation controller 110. In the implementation shown, the localizer 134 is an optical localizer and includes one or more optical sensors 140. The localizer 134 has a housing 138 comprising an outer casing that houses the one or more optical sensors 140. The optical sensors 140 may detect light signals, such as infrared (IR) signals and/or visible light signals. Localizer 134 may be mounted on an adjustable arm to position the optical sensors 140 with a field-of-view of the below discussed trackers that, ideally, is free from obstructions. The localizer 134 includes a camera controller 142 in communication with the optical sensors 140 to receive signals from the optical sensors 140. The camera controller 142 communicates with the navigation controller 110 through either a wired or wireless connection (not shown). In other implementations, the optical sensors 140 communicate directly with the navigation controller 110. Position and orientation signals and/or data are transmitted to the navigation controller 110 for purposes of tracking objects. The computer cart assembly 106, display 120, 121, and camera unit 136 may be like those described in U.S. Pat. No. 7,725,162 B2 to Malackowski, et al. issued on May 25, 2010, entitled “Surgery System,” the disclosure of which is hereby incorporated by reference. The navigation controller 110 is loaded with software that converts the signals received from the camera unit 136 into data representative of the position and orientation of the objects being tracked. The navigation controller 110 includes a navigation processor. It should be understood that the navigation processor could include one or more processors to control operation of the navigation controller 110. The processors may be any type of microprocessor or multi-processor system. The term processor is not intended to limit the scope of any implementation to a single processor.

Navigation system 104 is operable with a plurality of trackers 144, 146, 148, also referred to herein as trackers. In the illustrated implementation, one tracker 144 may be firmly affixed to a first portion of the patient and another tracker 146 may be firmly affixed to another portion of the patient. In some examples, trackers 144, 146 are firmly affixed to sections of bone (e.g., skull) in an implementation. The trackers 144, 146 may be mounted to other tissue types or parts of the anatomy. A tool tracker 148 may be coupled to the surgical tool 109 at any suitable location. The tool tracker 148 may be integrated into the surgical tool 109 during manufacture or may be separately mounted to the surgical tool 109 (or to an end effector attached to the manipulator of which the surgical tool 109 forms a part) in preparation for surgical procedures. The working end of the surgical tool 109, which is being tracked by virtue of the tool tracker 148, may be referred to herein as an energy applicator, and may be a rotating bur, saw, router, reamer, impactor, electrical ablation device, cut guide, tool holder, probe, or the like.

In one implementation, optical sensors 140 of the localizer 134 receive light signals from the trackers 144, 146, 148. In one example, the trackers 144, 146, 148 are passive trackers. In this implementation, each tracker 144, 146, 148 has at least three passive tracking elements or markers (e.g., reflectors) for transmitting light signals (e.g., reflecting light emitted from the camera unit 136) to the optical sensors 140. In other implementations, active tracking markers may be employed. The active markers may be, for example, light emitting diodes transmitting light, such as infrared light. Active and passive arrangements are possible. The camera unit 136 receives optical signals from the trackers 144, 146, 148 and outputs to the navigation controller 110 signals relating to the position of the tracking markers of the trackers 144, 146, 148 relative to the localizer 134. Based on the received optical signals, the navigation controller 110 generates data indicating the relative positions and orientations of the trackers 144, 146, 148 relative to the localizer 134. These relative positions may be displayed on the clinical application CA as graphical representations for surgical guidance.

In another implementation, the navigation system 104 and/or the localizer 134 are radio frequency (RF) based. For example, the navigation system 104 may comprise an RF transceiver coupled to the navigation controller 110. Here, the trackers 144, 146, 148 may comprise RF emitters or transponders, which may be passive or may be actively energized. The RF transceiver transmits an RF tracking signal, and the RF emitters respond with RF signals such that tracked states are communicated to (or interpreted by) the navigation controller 110. The RF signals may be of any suitable frequency. The RF transceiver may be positioned at any suitable location to track the objects using RF signals effectively. Furthermore, examples of RF-based navigation systems may have structural configurations that are different than the navigation system 104 illustrated throughout the drawings.

In other examples, the navigation system 104 and/or localizer 134 are electromagnetically (EM) based. For example, the navigation system 104 may comprise an EM transceiver coupled to the navigation controller 110. Here, the trackers 144, 146, 148 may comprise EM components attached thereto (e.g., various types of magnetic trackers, electromagnetic trackers, inductive trackers, and the like), which may be passive or may be actively energized. The EM transceiver generates an EM field, and the EM components respond with EM signals such that tracked states are communicated to (or interpreted by) the navigation controller 110. The navigation controller 110 may analyze the received EM signals to associate relative states thereto. Here too, examples of EM-based navigation systems may have structural configurations that are different than the navigation system 104 illustrated throughout the drawings.

In other examples, the navigation system 104 and/or the localizer 134 could be based on one or more other types of tracking systems. For example, an ultrasound-based tracking system coupled to the navigation controller 110 could be provided to facilitate acquiring ultrasound images of markers that define trackable features on the tracked objects such that tracked states are communicated to (or interpreted by) the navigation controller 110 based on the ultrasound images. By way of further example, a fluoroscopy-based imaging system (e.g., a C-arm) coupled to the navigation controller 110 could be provided to facilitate acquiring X-ray images of radio-opaque markers that define trackable features such that tracked states are communicated to (or interpreted by) the navigation controller 110 based on the X-ray images.

Furthermore, in some examples, a machine-vision tracking system including a vision camera may be coupled to the navigation controller 110 and could be provided to facilitate acquiring 2D and/or 3D machine-vision images of structural features that define trackable features such that tracked states are communicated to (or interpreted by) the navigation controller 110 based on the machine-vision images. The machine vision system may be integrated into the camera unit 136, optionally in combination with infrared sensors. The machine vision system may create depth maps and may detect objects with or without trackers. The machine vision system may detect patterns, shapes, colors, computer-codes, tracking geometries, or the like. The machine vision system may also be coupled to a moveable arm or a robotic arm.

Various types of tracking and/or imaging systems could define the localizer 134 and/or form a part of the navigation system 104 without departing from the scope of the present disclosure. Furthermore, the navigation system 104 and/or localizer 134 may have other suitable components or structure not specifically recited herein, and the various techniques, methods, and/or components described herein with respect to the optically-based navigation system 104 shown throughout the drawings may be implemented or provided for any of the other examples of the navigation system 104 described herein. For example, the navigation system 104 may utilize solely inertial tracking and/or combinations of different tracking techniques, sensors, and the like. Other configurations are contemplated.

Based on the position and orientation of the trackers 144, 146, 148 and previously loaded data, the navigation controller 110 may determine the position of the working end of the surgical tool 109 (e.g., the centroid of a surgical bur, needle, probe, etc.) and/or the orientation of the surgical tool 109 relative to the tissue against which the working end is to be applied.

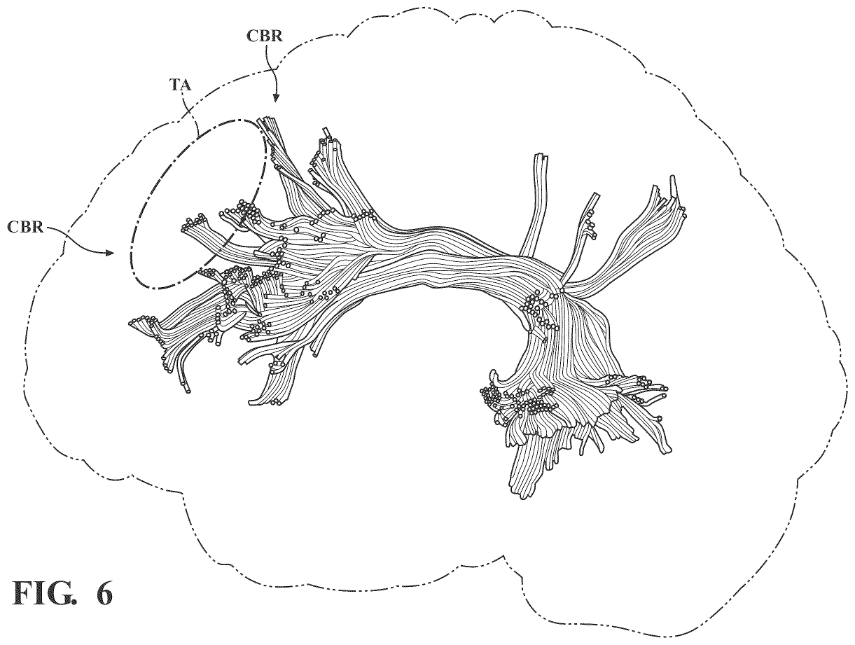

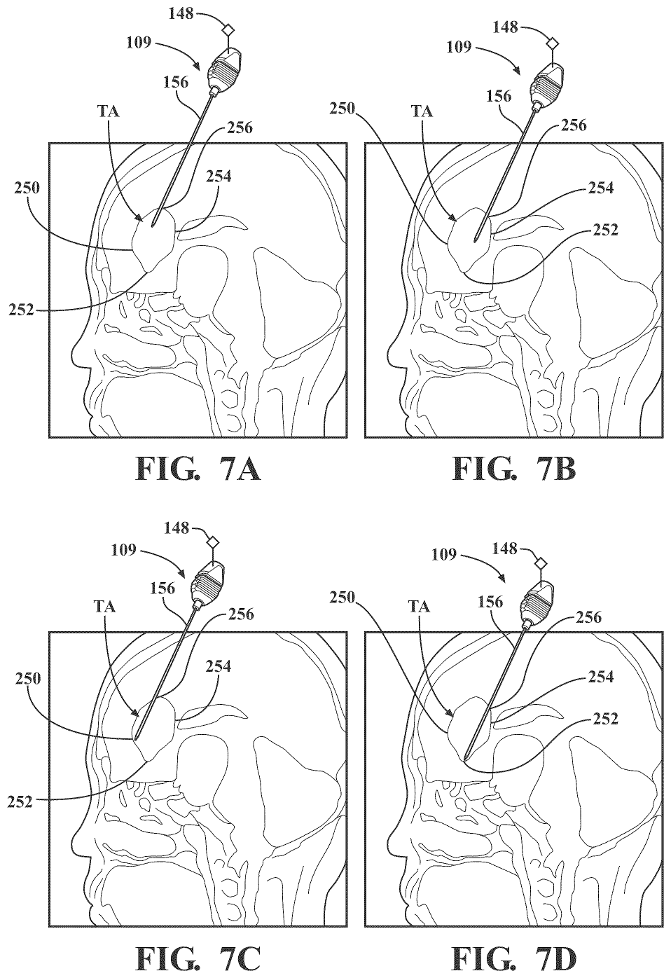

The virtual cutting boundary VB may be defined within a virtual model of the anatomy (e.g., cranium, brain), or separately from the virtual model. Example virtual boundaries are shown in FIGS. 6A-6D and 7A-7D. The virtual cutting boundary may be represented as a mesh surface, constructive solid geometry (CSG), voxels, or using other boundary representation techniques. The surgical tool 109 may be used to cut away material of the target anatomy to remove problematic tissue from the critical brain regions CBR.

The navigation controller 110 also generates image signals that indicate the relative position of the working end to the tissue. These image signals are applied to the displays 120, 121. The displays 120, 121, based on these signals, generate images on the clinical application CA that allow the surgeon and staff to view the relative position of the working end to the target anatomy of the target site.

Prior to the start of the intraoperative procedure, preoperative images of the target anatomy may be generated (or of other portions of the anatomy in other implementations). The preoperative images may be stored as two-dimensional or three-dimensional patient image data in a computer-readable storage device, such as memory within the navigation system 104. The patient image data may be based on X-ray scans, MRI scans, single-photon imaging, and/or computed tomography (CT) scans of the patient's anatomy. The patient image data may then be used to generate two-dimensional images or three-dimensional models of the patient's anatomy. The pre-operative data and models may be used for purposes of surgical planning and intraoperative guidance. For example, the surgical plan (e.g., tool path, resection volume, or boundaries VB), may be planned relative to the virtual model. The virtual model and surgical plan may then be registered to the anatomy using any appropriate registration technique.

The working end of the surgical tool 109 has its own coordinate system. In some implementations, the surgical tool 109 comprises a handpiece and an accessory that is removably coupled to the handpiece. The accessory may be referred to as the energy applicator and may comprise a probe, a bur, an electrosurgical tip, an ultrasonic tip, or the like. Thus, the working end of the surgical tool 109 may comprise the energy applicator.

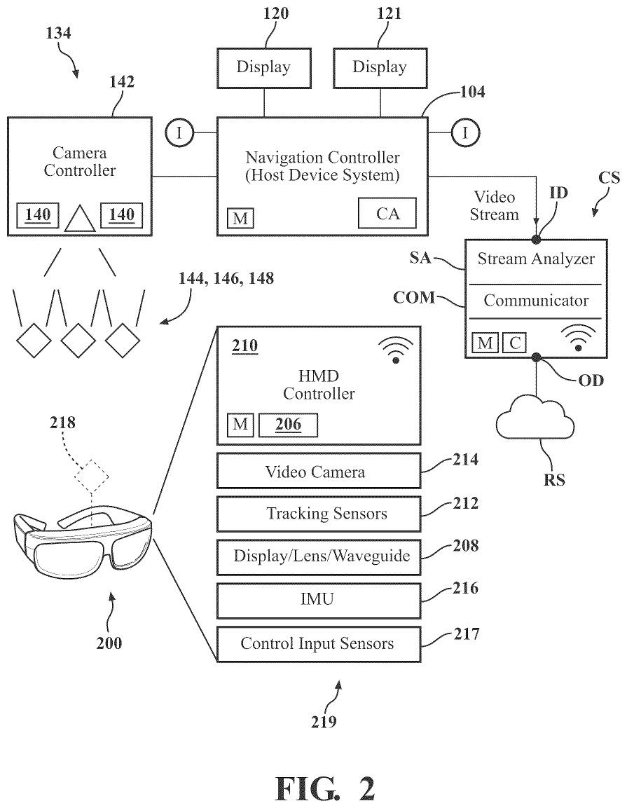

Referring to FIG. 2, a localization engine is a software module that may be considered part of the navigation system 104. Components of the localization engine run on navigation controller 110. Localization engine receives as inputs the signals from the localizer 134 and, in some implementations, signals from the tracker controller. Based on these signals, the localization engine may determine the pose (position and/or orientation) of the tracker coordinate systems in the localizer coordinate system. Based on the same signals received for the tracker 148, the localization engine determines the pose of the tracker coordinate system in the localizer coordinate system.

The localization engine forwards the signals representative of the poses of trackers 144, 146, 148 to a coordinate transformer. Coordinate transformer is a navigation system software module that runs on navigation controller 110. Coordinate transformer references the data that defines the relationship between the preoperative images of the patient and the patient trackers 144, 146. Coordinate transformer may also store the data indicating the pose (position and/or orientation) of the working end of the surgical tool 109 relative to the tracker 148.

During the procedure, the coordinate transformer may receive data indicating the relative poses of the trackers 144, 146, 148 to the localizer 134. Based on the previously loaded data, the coordinate transformer may generate data indicating the relative positions and orientations of the coordinate system and the anatomy coordinate systems. As a result, coordinate transformer generates data indicating the position and orientation of the working end of the surgical tool 109 relative to the tissue (e.g., target anatomy) against which the working end is applied. Image signals representing this data are forwarded to displays 120, 121 enabling the surgeon and staff to view this information.

While the example surgical system 100 has been described with reference to the Figures, the surgical system 100 is not intended to be limited to what is specifically shown and described. For example, the surgical system 100 may include a robotic manipulator (not shown). Other systems are contemplated without departing from the scope of the disclosure.

### II. Head Mounted Device

Referring back to FIGS. 1-3, one or more head-mounted devices (HMDs) 200 may be incorporated into the surgical system 100. The HMD may be employed to enhance visualization before, during, and/or after surgery. The HMD 200 is an extended reality device, which may include aspects of augmented reality, mixed reality, virtual reality, and the like. The HMD 200 may be used to visualize the same objects previously described as being visualized on the displays 120, 121, and may also be used to visualize other objects, features, instructions, warnings, etc. In some examples, the HMD 200 may be worn by a surgeon and/or members of the surgical team. The HMD 200 may be used to assist with visualization of the volume of material to be cut from the patient, to help visualize the size of implants and/or to place implants for the patient, to assist with registration and calibration of objects being tracked via the navigation system 104, to see instructions and/or warnings, among other uses as described further below. In some examples, such as shown in FIGS. 1-3, the HMD 200 may be worn by a patient to monitor and analyze physical and cognitive abilities, which will be described further below.

The HMD 200 has a display 208 onto which computer-generated content may be displayed onto a real-world view. In the implementation described herein, the HMD 200 provides on the HMD display 208 a computational holographic/superimposed/overlay of computer-generated content over the real-world view. In one example, the real-world view is acquired by a video camera 214 attached to the HMD. The video camera 214 produces a live video stream of the real-world and the computer-generated content may be combined into video stream of the real-world. In such instances, the HMD display 208 may include one or more high-resolution displays positioned in front of the user's eyes. The HMD display 208 may be opaque in such scenarios.

In other implementations, the HMD 200 may implement natural see-through techniques whereby the HMD display 208 is implemented as a transparent lens/visor/waveguide provided between the user's eyes and the real-world. The real-world view is acquired naturally by the user's eyes and the computer-generated content is provided on the transparent lens/visor/waveguide. Such see-through techniques may include a diffractive waveguide, holographic waveguide, polarized waveguide, reflective waveguide, or switchable waveguide.

The HMD 200 includes a support structure 202, which may be head-mountable in the form of an eyeglass or glasses, headwear or headset, or eyewear (such as a digital contact lens or lenses). The HMD 200 may include additional headbands or supports to hold the HMD 200 on the user's head. Although not shown, it is contemplated that instead of the HMD 200, an extended reality display screen, such as a monitor, tablet, or hand-held display may be used, which may include similar hardware and capabilities as the described HMD 200.

The HMD 200 may include an HMD controller 210. The HMD controller 210 may include a content generator 206 that generates the computer-generated content (also referred to as virtual images) and that transmits those images to the user through the HMD display 208. The HMD controller 210 controls the transmission of the computer-generated content to the HMD display 208. The HMD controller 210 may be a separate computer, located remotely from the support structure 202 of the HMD 200, or may be integrated into the support structure 202 of the HMD 200. The HMD controller 210 may be a laptop computer, desktop computer, microcontroller, or the like with memory, one or more processors (e.g., multi-core processors), input devices I, output devices (fixed display in addition to HMD 200), storage capability, etc.

The HMD 200 may include a plurality of tracking sensors 212 that are in communication with the HMD controller 210. In some cases, the tracking sensors 212 are provided to establish a global coordinate system for the HMD 200, also referred to as an HMD coordinate system. The HMD coordinate system is established by these tracking sensors 212, which may comprise camera sensors or other sensor types, in some cases combined with IR depth sensors, to layout the space surrounding the HMD 200, such as using structure-from-motion techniques or the like. The HMD 200 may also comprise a photo/video camera 214 in communication with the HMD controller 210. The camera 214 may be used to obtain photographic images or video with the HMD 200, which may be useful in identifying objects or markers attached to objects, as will be described further below. The HMD 200 may comprise an inertial measurement unit IMU 216 in communication with the HMD controller 210. The IMU 216 may comprise one or more 3-D accelerometers, 3-D gyroscopes, and the like, to assist with determining a position and/or orientation of the HMD 200 in the HMD coordinate system or to assist with tracking relative to other coordinate systems. The HMD 200 could have a speaker to generate a sound or vibrate to provide an indication to the HMD user of a warning or other information of relevance.

The HMD 200 may also comprise input sensors 217. In one example, the input sensors 217 are configured to recognize gestures or eye-based movements from the patient. In some examples, gestures or eye-based movements may be voluntary, and in other examples, may be involuntary. When detecting hand movements, the input sensors 217 are able to sense the patient's hands, fingers, or other objects for purposes of determining the patient's movement, and controlling the HMD 200, HMD controller 210, and/or navigation controller 110 accordingly. In some examples, the hand gestures may be detected below the HMD 200 or may be detected by the camera 214 in front of the HMD 200. The input sensors 217 to detect gestures may include one or more cameras, infrared sensors, motion sensors, or the like. Hand movement may include any type of hand or finger motion, including but not limited to: pinching, pointing, swiping, circling, grasping, twisting, or the like.

When detecting eye movements, the input sensor 217 is able to sense the patient's eye position, motion, dwell time (stare), gaze and the like, for purposes of monitoring and determining the patient's response and controlling the HMD 200, HMD controller 210, and/or navigation controller 110, accordingly. Eye movements may be detected using an eye-tracker that is positioned to face the patient's eyes, e.g., in front of the HMD display 208. Eye movements may be monitored to evaluate physical and/or cognitive abilities. In some examples, eye movements may be monitored as the patient receives commands from the HMD 200 to move their eyes in a particular way, including but not limited to: selecting an object, moving an object, following an object, or the like. In one example, the patient may select a computer-generated object displayed by the HMD 200 by staring at the object continuously for a threshold amount of time. The HMD 200 may also monitor and analyze auditory patient responses with input sensors 217 through a microphone. The HMD controller 210 may process the sounds from the patient and control the HMD display 208 in response.

Any of the described components of the HMD 200 that may sense information or process sensed information (including but not limited to, the HMD controller 210, the video camera 214, tracking sensors 212, IMU 216, and/or input sensors 217) may be understood as being part of a “sensing system” of the HMD 200. The sensing system is identified by numeral 219 in FIG. 2.

The HMD 200 may be registered to one or more objects used in the operating room, such as the tissue being treated, the surgical tool 109, the manipulator, the trackers 144, 146, 148, the localizer 134, and/or the like. In one implementation, a local coordinate system is associated with the HMD 200 to move with the HMD 200 so that the HMD 200 is fixed in a known position and orientation in the HMD coordinate system. The HMD 200 may utilize the tracking sensors 212 to map the surroundings and establish the HMD coordinate system. The HMD 200 may then utilize the camera 214 to find objects in the HMD coordinate system. In some implementations, the HMD 200 uses the camera 214 to capture video images of markers attached to the objects and then determines the location of the markers in the local coordinate system of the HMD 200 using motion tracking techniques and then converts (transforms) those coordinates to the HMD coordinate system.

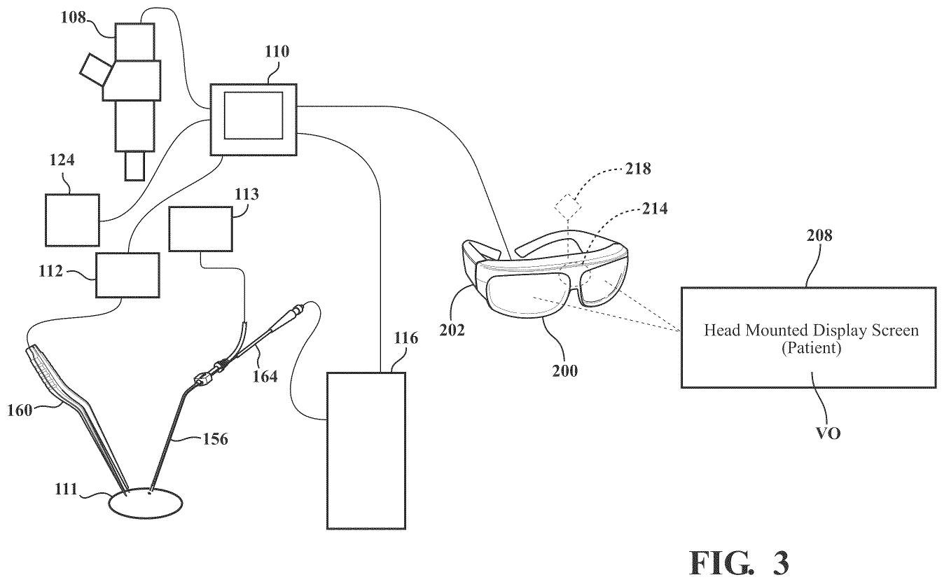

In another implementation, a separate HMD tracker 218 (see FIGS. 2 and 3), similar to the trackers 144, 146, 148, could be mounted to the HMD 200 (e.g., fixed to the support structure 202). The HMD tracker 218 may have its own HMD tracker coordinate system that is in a known position/orientation relative to the local coordinate system of the HMD 200. Alternatively, the tracker coordinate system could be calibrated to the local coordinate system using calibration techniques. The localizer 134 could then be used to track movement of the HMD 200 via the HMD tracker 218 and transformations could then easily be calculated to transform coordinates in the local coordinate system to the localizer coordinate system, the target anatomy coordinate system, or other coordinate system(s).

During use, for example, the localizer 134 and/or the navigation controller 110 may send data on an object to the HMD 200 so that the HMD 200 knows where the object is in the HMD coordinate system and may display an appropriate content in the HMD coordinate system. Once registration is complete, then the HMD 200 may be used to visualize computer-generated content in desired locations with respect to any objects in the operating room. Although these transforms have been described in detail, it is understood that the HMD 200 may operate without requiring any such transforms. The HMD 200 may display content without registering to the target anatomy, or any part of the surgical system 100.

### III. Techniques Involving the HMD

Having introduced the system 100 and HMD 200 above, this section now describes various systems, methods, software, and techniques involving the HMD 200 including: (1) a “smart” view technique whereby relevant information is automatically provided on the HMD 200 at appropriate times and locations, (2) a compatibility or connectivity technique or module that enables the HMD 200 to be seamlessly integrated with any host system/device that provides information on a software application and (3) techniques for reducing latency in communication of video data to the HMD 200. The above list of features introduces a selection of concepts in a simplified form that are further described below. The above list is not intended to limit the scope of the claimed subject matter nor identify key features or essential features of the claimed subject matter.

### A. Intelligent Contextual Display of Virtual Objects on HMD

The concepts described herein further include the dynamic display of one or more virtual objects VO on the HMD display 208 at appropriate locations and at appropriate times to the patient. In one example, the virtual object(s) VO are specifically presented relative to the patient-calibrated pose of the view coordinate system, as described above. However, the techniques described herein are not limited to such. Certain techniques may not involve displaying the virtual object(s) VO relative to any described coordinate system, such as the HMD coordinate system.

As will be understood from the description herein, the described techniques provide a comprehensive configuration for data collection and analysis of patient responses, and through the HMD 200, such as displaying and/or verbally commanding the patient to answer a question or perform a task and recording the patient response. The described techniques may display information to the patient in order to elicit a response from the patient.

### 1. Virtual Objects

The one or more virtual objects VO are computer-generated and may be 2D or 3D objects or combinations thereof. The controller(s) may present, on the HMD display 208, the one or more virtual objects VO combined with, or overlaid onto, the real-world view.

The virtual objects VO may be a series of virtual objects VO that are presented one after the other. Multiple virtual objects VO may be simultaneously displayed. The virtual objects VO may include parent and sub-objects or nested virtual objects. The virtual object(s) VO may be translated and/or rotated by the patient when the patient is responding to a command through the HMD 200 using the control inputs (such as gaze and gesture) and sensing system 219, as described above. Similarly, the patient may also move the virtual object(s) VO closer to or further away by moving the virtual object(s) VO towards their eyes or away from their eyes, respectively. In doing so, the size or the virtual object(s) VO may respectively increase or decrease in size depending on the magnitude and direction of the movement. Furthermore, in some examples, any of the virtual object(s) VO described herein may have opacity or transparency. When transparent or semi-transparent, the real-world view may be seen through the virtual object VO. Certain real-world objects, such as the patient's hand, may take priority over any virtual object VO. In one example, when the hand is detected by the HMD 200, the hand may be displayed in front of the virtual object VO.

The virtual object(s) VO may display any information related or relevant to the surgeon, patient, or surgical procedure. The surgical information may, but need not, be related to the process of actually performing surgery. The surgical information may be pre-operative, intraoperative, or post-operative surgical information. Examples of surgical information include but are not limited to: patient information, patient testing and evaluation information, medical images (e.g., CT scan or volume, X-rays, etc.), surgical guidance information (e.g., tool interaction with target site), surgical planning information, an anatomical model, an implant model, a cut plan, a resection plan or volume, a virtual boundary VB or cutting boundary, surgical tool information, operating room or tool setup information, surgical step information, clinical application information, surgical alerts, notifications or warnings, and the like. In some examples, such as described further below, the virtual objects VO may be configured as a patient test to elicit a response from a patient for evaluating anatomical and/or cognitive functions. The surgical information may be a step of the surgical procedure. The step of the surgical procedure may include but are not limited to: a pre-operative planning step, pre-operative evaluation of a patient, an operating room setup step, an anatomical registration step, an intra-operative planning step, an anatomical preparation step, or a post-operative evaluation step. The virtual object(s) VO may be a 3D and/or 2D surgical object relevant to any of the above information. Examples of such virtual objects VO include but are not limited to: virtual screens, windows, or information panels, tests, and/or commands to elicit a response, a 3D model of a bone, a 3D model of an implant, and a 3D surgical plan.

In one example, the virtual object(s) VO may be a virtual information panel. The virtual information panel may include 2D and/or 3D graphical elements, as well as text. The virtual information panel may be configured to present any relevant information in a “window” style format. The virtual information panel may have minimal thickness to emulate a flat object, such as a flat screen.

2. Recognition of Surgical information

In one implementation, the HMD 200 may automatically display one or more virtual object(s) VO specifically in response to a recognition of surgical information. The surgical information may be recognized according to various implementations and from various sources, as will be described below.

a. Obtaining Surgical Information from Video Stream Analysis of Host System/Device Software Application

In one example, the surgical information may be identified and/or extracted from the navigation system 104 or any host system/device (e.g., in the operating room) that is configured to display a software application, such as the clinical application CA presented by the navigation system 104. Although the navigation system 104 and its clinical application CA is largely described herein as one example, the host system/device 104 and respective software application may take various forms. For example, the host system/device 104 and software application may include any of: an endoscopic system that operates a software application for the endoscopic system; an imaging system (e.g., CT scanner) that operates a software application for the imaging system; a (CORE) console that operates a software application for operation of powered instruments; a surgical robot that operates a software application for controlling the surgical robot, a hand-held tool 109 that operates a software application for controlling the hand-held tool 109, a surgical visualization system (e.g., arthroscope, ultrasound, laparoscope) that operates a software application for controlling the surgical visualization system, a surgical waste management system that operates a software application for controlling the surgical waste management system, a fluid management system that operates a software application for controlling the fluid management system, a sponge management system that operates a software application for controlling the sponge management system, a patient support apparatus that operates a software application for controlling the patient support apparatus, and the like.

Referring to FIGS. 1 and 2, the surgical system 100 may include a connectivity system or kit, CS, which communicates between the navigation system 104 (or host system/device) and the HMD 200 to identify or extract such surgical information and perform other functions. In one example, the connectivity system CS includes a computing system (C), and an input device (ID) and output device (OD) and memory (M) coupled to the computing system C. The input device ID is configured to receive a video stream of the software/clinical application from the host system/device (e.g., navigation system 104). For example, the connectivity system CS may receive a video stream of the clinical application CA from the navigation system 104. The input device ID may be coupled to the host system/device 104 using a wired input, such as a HMDI or DVI input. Conversion devices may be utilized to convert the format of the video stream (e.g., converting from DVI to HMDI for example). The computing system C is configured to automatically analyze and recognize information from the video stream of the software/clinical application. The computing system C may implement a stream analyzer SA to perform this function. In response to recognition of the information, the computing system C may generate the virtual object(s) VO related to the recognized information. The connectivity system CS may also include a communicator COM, which is configured to communicate with the HMD 200. The communicator COM may include any one or more devices that enable such communication. In one example, the communicator COM includes a wireless communication system, such as a Wi-Fi router, Bluetooth transmitter, or the like. The HMD 200 is configured to communicate using the chosen communication method provided by the connectivity system CS. The output device OD may be the communicator COM itself or the output device OD may be coupled to the communicator COM. The connectivity system CS may also be configured to receive any other type of data from the host system/device, such as control data, calibration data, or other information related to operation of the host system/device.

In some examples, the connectivity system CS may be integrated, in part or in whole, into the host system/device or navigation system 104. For example, the connectivity system CS may be implemented by the navigation controller 110 and the components of the connectivity system CS may be incorporated into the cart assembly 106. Also, the connectivity system CS may be integrated, in part or in whole, into the HMD 200.

As described above, the surgical information may be obtained from a host device/system, such as the navigation system 104. In the example of the navigation system 104, the clinical application CA may be presented on one or more displays 120, 121 of the navigation system 104. Through connection to the connectivity system CS, the computing system C may obtain the video stream of the clinical application CA. In effect, the computing system C may obtain video, in real-time, and corresponding to imagery, text or information is configured to be presented by the clinical application CA. The clinical application CA may be presented on the displays 120, 121 of the navigation system 104 while the video stream is obtained by the computing system C and the video stream may reflect real-time user modifications to the clinical application CA. However, it is not always required that the clinical application CA be presented (e.g., if the display 120, 121 is turned off). The computing system C utilizes the stream analyzer SA to analyze and recognize the surgical information from the video stream of the clinical application CA. Notably, the techniques described herein may be performed by the navigation system 104 (or host system/device) in situations where the components of the connectivity system CS are integrated into the navigation system 104 (or host system/device) instead of being a stand-alone device. In such instances, the computing system C may be understood as including the navigation controller 110 and its respective components, or any components native to the host system/device.

In one example, the surgical information may be identified and/or extracted from the clinical application CA and communicated through connectivity system CS to queue the HMD 200 to display one or more evaluation prompts to a patient. For example, when the clinical application CA has indicated that a surgeon has switched from a first surgical tool, such as probe 158 to a second surgical tool, such as powered forceps 160, and has moved the bipolar forceps relative to the target anatomy TA, HMD 200 may display an evaluation to a patient to monitor the patient's anatomical and/or cognitive functions during the procedure. In another example, the surgical information may be identified and/or extracted from the navigation system 104 and communicated through the connectivity system CS to queue the HMD 200 to display one or more evaluation prompts to the patient. Additionally, and/or alternatively, the surgical information may be communicated directly and/or indirectly throughout the system by BUS communication.

In one implementation, the computing system C utilizes the stream analyzer SA to recognize the surgical information by automatically identifying text presented by the clinical application CA. The stream analyzer SA may utilize any text recognition algorithm to perform this function, such as optical character recognition OCR, visual text recognition, scene text recognition, natural language processing NLP, any combination thereof, and the like.

The computing system C may utilize the stream analyzer SA to recognize the surgical information by automatically identifying imagery or graphics presented by the clinical application CA. The stream analyzer SA may utilize any image recognition algorithm to perform this function, such as segmentation, bounding boxes, pattern recognition, shape modeling, machine learning models, deep learning, neural networks, convolutional neural networks, any combination thereof, and the like. Additionally, or alternatively, the computing system C utilizes the stream analyzer SA to recognize the surgical information by automatically identifying user inputs provided on the clinical application. Such inputs may include mouse movements or behavior, cursor selections, inputted text (e.g., using a keyboard), screen selections, icon selections, movement, or manipulation of graphical objects, such as scroll bars, up/down arrows, bone models, implants, and the like.

The surgical information identified or extracted by the computing system C may include any information that may be relevant to the surgeon, patient, or surgical procedure. The surgical information may, but need not, be related to the process of actually performing surgery. The surgical information may be pre-operative surgical information. Alternatively, surgical information may include post-operative information, such as reports, etc. Examples of surgical information include but are not limited to: patient information, medical images (e.g., CT scan or volume, X-rays, etc.), surgical guidance information (e.g., tool interaction with target site), surgical planning information, an anatomical model, an implant model, a cut plan, a resection plan or volume, a virtual boundary VB or cutting boundary, surgical tool information, operating room or tool setup information, surgical step information, clinical application information, surgical alerts, notifications or warnings, testing and/or evaluation prompts, and the like.

In some implementations, certain regions of the screen may be monitored from the video stream to detect surgical information that subsequently triggers monitoring or detection of another region of the screen. For example, the detection of specific text on the screen may trigger clipping or reproduction of a graphical part of the screen for display on the virtual object(s) VO.

Other methods of obtaining surgical information from the host system/device software/clinical application are contemplated. Any of the techniques described above may be used individually or in combination.

b. Obtaining Surgical Information from Camera or Other Source(s)

The example surgical system 100 described has various camera sources. These camera sources may include the HMD camera 214 and the camera (visible light or machine vision camera) of the navigation system 104. Additional camera sources may include a camera source attached to the tool 109. Such tool cameras include, but are not limited to: a scope, an endoscope, a laparoscope, an arthroscope, and a microscope. Other camera sources may be in the operating room, such as other HMDs 200, a camera attached to the manipulator, or a dedicated (standalone) camera utilized for viewing a separate display device. Any of these cameras constitute a camera source for purposes of this description.

Any camera source may detect surgical information. Such surgical information may be detected at the target site or elsewhere. The surgical information detected by the camera source may be processed by any suitable controller or computing system, depending on the system configuration. Such controllers/computing systems may include but are not limited to, the camera controller 142, the navigation controller 110, the computing system C, the tool controller, or the HMD controller 210.

Surgical information detectable by the camera source may include any information that may be relevant to the surgeon, patient, or surgical procedure. The surgical information may, but need not, be related to the process of actually performing surgery. The surgical information may be pre-operative or post-operative surgical information. Examples of surgical information detectable by the camera source include but are not limited to: location and/or detection of any surgical object (such as the bone, tracker, surgical tool, robot or end effector, sensitive tissues, retractors, surgical table, imaging device, etc.), tool identification, anatomy information, surgical guidance information (e.g., tool interaction with target site), interaction between tools, amount of bone removed or needed to be removed, tool path, tool calibration, tool or component installation, surgical planning information, identification of an obstruction to a tool, line-of sight obstructions, surgeon ergonomics or posture, and the like.

The surgical information detectable by the camera source may also include any information presented by the clinical application CA on the navigation system 104 display 120, 121. For example, by the HMD user looking at the display 120, 121, the HMD camera 214 may capture a live-video stream of the clinical application CA. In so doing, the HMD camera 214 may detect the content of the clinical application CA on the displays 120, 121 and identify or extract surgical information. The HMD 200 may analyze the contents of the video stream in a manner similar to that described above (e.g., by identifying or extracting text and/or images). The HMD 200 may perform this function using the HMD controller 210 and/or the computing system C and stream analyzer SA.

Surgical information may also be detectable by other sensing features of the HMD 200, including any component of the sensing system 219 of the HMD 200, including the microphone, the IMUs 216, the tracking sensors 212, and/or the input sensors 217. For example, the microphone may detect an alert, sound, or message outputted by the navigation system 104, such as a chime to indicate that a step has been successfully performed. The HMD 200 microphone may detect this chime and process the chime as input surgical information. Other methods of obtaining surgical information from such other sources are contemplated. Any of the techniques described above may be used individually or in combination.

### 2. Virtual Object Presentation

As described above, surgical information may be identified or extracted in various forms and by various systems, such as by analyzing the video stream of the software/clinical application and/or by obtaining input from any other source(s) such as cameras. The surgical information may be processed by any suitable controller or computing system, depending on the system configuration. Such controllers/computing systems may include but are not limited to, the camera controller 142, the navigation controller 110, the connectivity system CS, the computing system C, the tool controller, or the HMD controller 210. Whatever the applicable system used to process the surgical information is referred to in this section as the “controller(s).”

Upon processing of the surgical information, video data and/or command signals are communicated by the controller(s) to the HMD 200 for presentation. The HMD 200 may automatically display or be commanded to display, one or more virtual object(s) VO specifically in response to recognition of such surgical information. The software/clinical application CA may or may not be presented on the host system/device display concurrently while the virtual object VO is presented on the HMD display 208.

During or after the procedure, the controller(s) may transmit, to the remote server RS, any information from the system 100, such as information recognized from the video stream or any contents that are displayed on the HMD 200. These contents may include a video stream transmitted to the HMD 200, a video stream produced by the HMD 200, any text or graphics detected within the video stream and/or virtual objects VO that were displayed on the HMD 200. Other information may be logged, such as user inputs or behavior, system performance data, data transmission or performance, etc. The information may be transmitted for post-operative data analytic purposes or for improving future uses of the HMD 200. The remote server RS may be a cloud server or any suitable type of remote server. Multiple HMDs in the same facility or from multiple locations may communicate to the remote server RS. The remote server RS may include software for analyzing the information from the multiple HMDs to perform any of the described features. The remote server RS may also communicate software updates, calibration settings, or any other information described herein to any HMD 200.

### 3. Example Method(s) of Patient Testing and Evaluation Technique(s) Involving the HMD

As discussed above, an awake craniotomy may be done in cases when a tumor (target anatomy TA) is close to regions of the brain that control language, cognition, sensation, and body movement. By keeping the patient awake, the surgical team may precisely map important areas of the brain to avoid during the surgery to preserve the patient's language, sensory, and motor abilities. Before any brain tissue is removed, the surrounding critical brain regions may be target anatomy TA and are tested for their role in critical brain functions. Awake brain mapping involves asking the patients to do various tasks while specific parts of the brain are stimulated with probe 158. The patient assists the process by answering questions and/or responding to prompts (e.g., counting, identifying pictures, reading, etc.), to identify which parts of the brain are critical for functions like language, body movement, hearing, sight, touch, etc. These regions may be referred to as critical brain regions CBR. By mapping the brain's critical areas relative to the target anatomy TA, the surgeon may be able to remove as much of the tumor as possible while preserving critical brain functions.

The HMD 200 may be connected with the navigation system 104. As the navigation system 104 tracks the surgical tool 109, the HMD 200 may produce specific virtual objects VO and/or auditory commands to the patient based on the position of the surgical tool 109 relative to the target anatomy TA and critical structures (critical brain regions CBR). In some examples, the HMD 200 may automatically provide outputs to the patient in order to elicit a response from the patient based on the position of the surgical tool 109.

Turning to FIGS. 5-6 and 8A-8D, virtual boundaries VB may be established based on target anatomy TA relative to the critical brain regions CBR, which may be identified using patient images (FIGS. 5 and 6). FIGS. 5 and 6 illustrate examples of patient images utilizing CT, MRI, and/or single-photon imaging. As described above, the surgical system 100 generates a 3D model and registers the 3D model with the navigation system 104.

FIG. 5 is an example of a patient image which shows several views of the brain with a tumor (target anatomy TA). FIG. 6 is one example of a single-photon emission image which may be used to map neural pathways. Using the patient images of FIG. 5 and/or FIG. 6, the surgical system 100 registers the images with the navigation system 104. As described above, the navigation system 104 tracks the patient with patient trackers 144, 146, and tracks surgical instruments 109 with instrument tracker 148.

In some examples, before resection to remove target anatomy TA, a patient baseline may be established to analyze patient responses intraoperatively. Put differently, the baseline establishes normal functionality of the patient. The establishment of baseline responses may be conducted pre-operatively and/or intraoperatively. In some examples, when the baseline responses are established preoperatively, the HMD 200, a surgical specialist, or both, may prompt the patient to respond to visual and/or auditory prompts/stimulus so that the patient's “normal” functionality is monitored and recorded. For example, the visual and/or auditory prompts/stimulus may be to identify a picture, solve a math equation, identify a sound, follow an object with their eyes, move their appendages, the like, or a combination thereof. The patient responses are recorded and analyzed by the surgical system 100, including the HMD 200. The patient responses that are used to establish the patient's baseline (e.g., “normal” functions of the patient before brain surgery), so that there is data to compare intraoperative patient responses against in order to determine whether a critical brain region CBR has been or will be negatively affected if resected.

Similarly, to establish baseline activity intraoperatively, the patient may receive instructions from a surgical specialist, an HMD 200, or both. In one example, the instructions are provided through the HMD 200. The instructions may function to elicit a response from the patient in order to establish a baseline of the patient's senses and cognitive functions. To establish the baseline, the patient responds to the prompts/stimuli provided by the HMD 200. The response of the patient may be recorded by the HMD 200 through the sensing system 219 (described above with reference to FIG. 2). In one non-limiting example, the HMD 200 may command a patient to follow a virtual object VO across the HMD 200 with their eyes. In another non-limiting example, the HMD 200 may command the patient to identify a virtual object VO that is presented on the HMD display screen 208. In another non-limiting example, the HMD 200 may present the patient with questions to answer or sounds to identify. In another non-limiting example, the HMD 200 may command the patient to move their fingers. It is contemplated that the patient may be instructed to perform one or more actions involving one or more senses and/or cognitive ability which may be recorded by the sensing system 219 of the HMD 200. This process may happen pre-operatively and/or intraoperatively. Further, it is contemplated that this process may also be used to evaluate a patient post operatively.

After the patient images are registered with the navigation system 104, and a baseline is established relating to “normal” patient responses, verifying the location of the critical brain regions CBR may be determined through mapping. To map the brain, a probe 158 may be used intraoperatively to verify the location of the critical brain regions CBR and ensure that during removal of the target anatomy TA that desirable tissue (critical brain regions CBR) will not be affected. Mapping may function to verify the location of critical brain regions CBR, since the brain is not rigidly fixed to the body and may move before and/or during the procedure from the location of the brain when the patient images were acquired.

In some examples, as briefly mentioned above, the probe 158 may be a neuro stimulator. The probe 158 may function to stimulate sections of a patient's anatomy (e.g., brain) with an electrical stimulus. In combination with the patient images, the probe 158 may function to verify the critical brain regions CBR and the patient's functionality associated with the critical brain regions. It is contemplated that the probe 158 may be capable of assisting with mapping portions of the patient's anatomy by probing the brain and stimulating the area with the electrical stimulus to establish and/or verify where the critical brain regions CBR are located.

Turning to FIGS. 7A-7D, the probe 158 may be used to locate and/or verify the location of critical brain regions CBR relative to the target anatomy TA. In some examples, to locate and/or verify the position of the critical brain regions CBR, a surgeon may use probe 158 in conjunction with HMD 200 to evaluate brain tissue and the functions that the brain tissue controls. In some examples, such as in FIGS. 7A-7D, the probe 158 is moved from location to location. As the probe 158 is moved from location to location, the electrical stimulus is applied to the critical brain regions CBR (shown as critical brain areas 250, 252, 254, 256). As the electrical stimulus from the probe 158 is administered to the critical brain areas 250, 252, 254, 256, certain responses are anticipated by the surgical system 100, which confirm the location of the critical brain areas 250, 252, 254, 256.

Instructions are conveyed to the patient via the HMD 200 to elicit a response while the probe 158 applies a current to the brain tissue at a particular location, such as shown in FIGS. 7A-7D. The HMD 200 may vary in the types of prompts and/or tests presented to the patient to evaluate specific portions of the brain (e.g., critical brain regions CBR) that the surgical tool 109 is stimulating. In some examples, the probe 158 is used to check the tissue within the virtual boundary VB (established through the patient images) to determine whether removal of the target anatomy TA overlaps with critical brain regions CBR. In one example, the target anatomy TA is a brain tumor between the frontal lobe and of a patient (FIGS. 5-8D). In some examples, the preoperative patient images (described above) are registered with the navigation system 104, allowing the surgical tool 109 (probe 158) to be tracked (via tracker 148) relative to the patient, based on the patient image and the anatomy trackers 144, 146 to confirm the location of the surgical tool 109 relative to the critical brain regions CBR.

In some examples, while mapping the patient's brain, when stimulating a patient with surgical tool 109, the patient may respond to the stimulus applied to a portion of the brain. For example, probe 158 may be inserted into the frontal lobe (which controls cognitive function) and actuated to apply an electrical stimulus. In this example, during the mapping procedure, the patient is given prompts to respond to relating to cognitive function, so that when the probe 158 is actuated and applies a stimulus, the patient response is monitored to confirm the functionality of that particular area of the brain.

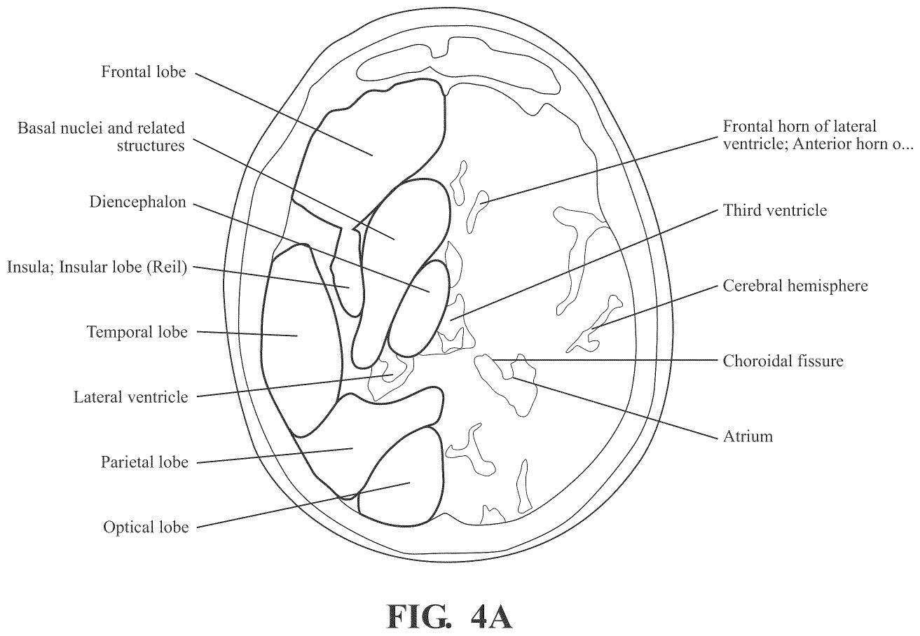

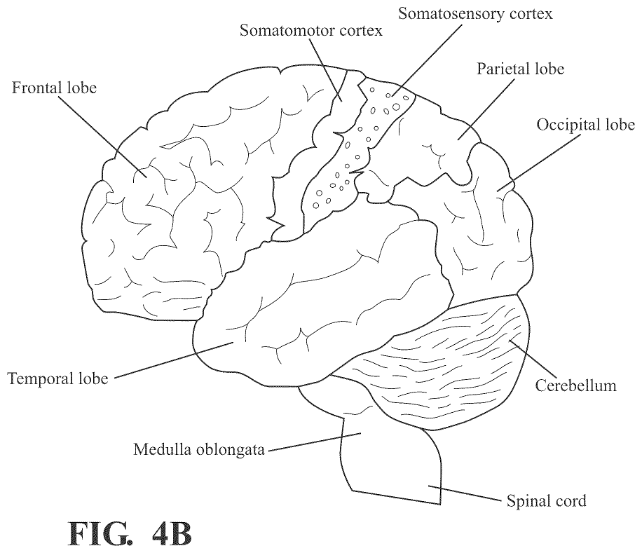

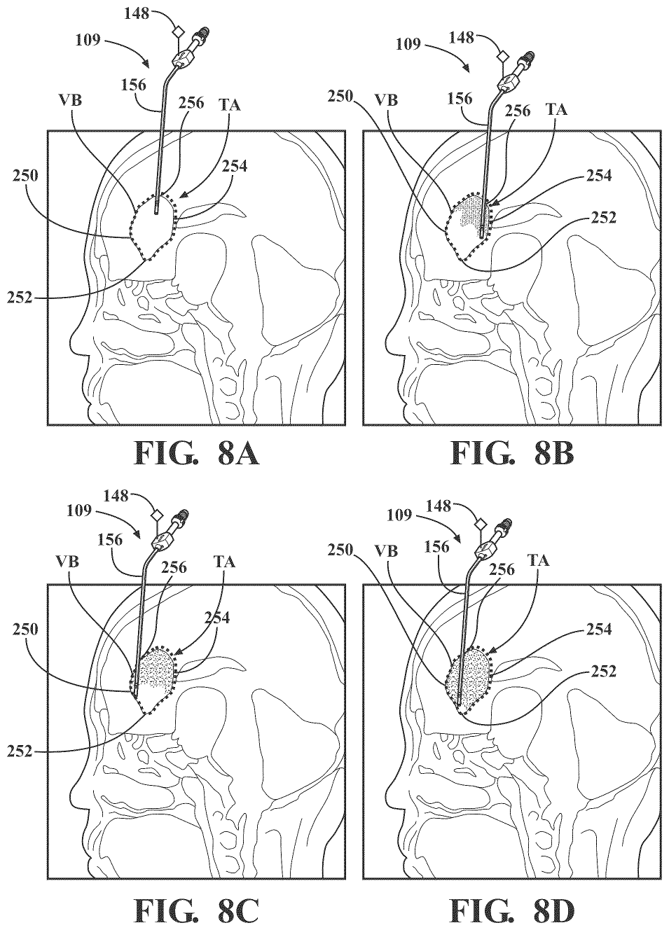

In some examples, as the probe 158 is moved, the HMD 200 may automatically change the types of prompts given to the patient to elicit a response based on the location of the probe 158 relative to critical brain regions CBR. As shown in FIGS. 7A-7D and 8A-8D, critical brain regions CBR are critical brain areas 250, 252, 254, 256. In some examples, the patient responses may be eye-based, hand-based, auditory, verbal, or a combination thereof. FIGS. 7A-7D show the probe 158 testing areas of the target anatomy TA relative to critical brain regions CBR. The probe 158 and the HMD 200 may work in conjunction to determine when critical tissue (critical brain areas 250, 252, 254, 256) is reached with the probe 158 by monitoring responses by the patient with the HMD 200. In one example, such as shown in FIG. 7A, the probe 158 is located near critical brain area 256, corresponding to the Somatomotor Cortex of the Primary Motor Cortex (See FIG. 4B), so the HMD 200 may prompt the patient to elicit responses relating to appendage movement, which is associated with critical brain area 256. Similarly in FIG. 7B, the probe 158 approaches critical brain area 254 associated with the Somasensory Cortex of the Primary Motor Cortex (See FIG. 4B), the HMD 200 provides prompts to the patient to elicit a response from the patient to monitor the patient's sense of touch. As seen in FIG. 7C, the probe 158 is approaching critical brain area 250 associated with the Frontal Lobe (see FIG. 4B), which causes HMD 200 to provide prompts and/or commands to the patient to elicit responses relating to cognitive function. FIG. 7D shows the probe 158 near critical brain area 252 also located at/near the Frontal Lobe. In one example, the HMD 200 may provide prompts and/or commands to elicit a different type of response from the patient relating to cognitive function associated with critical brain area 252 than the prompts and/or commands associated with the critical brain area at 250. For example, a first command may be given to the patient to elicit a first type of response from the patient, such as reciting the alphabet, when the probe 158 is near critical brain area 252, and a second command may be given to the patient to elicit a second type of cognitive response from the patient, such as reciting the patient's place of birth, when probe 158 is near critical brain area 250. In another example, the HMD 200 may provide prompts and/or commands to the patient to elicit the same type of cognitive response from the patient relating to cognitive function when the probe 158 is near critical brain area 252 and critical brain area at 250.

As previously described with reference to input sensors 217, the HMD 200 is able to sense the patient's eye position, motion, dwell time (stare), gaze, and the like. Similarly, the HMD 200 is able to sense the patient's hands, fingers, or other objects for purposes of determining the patient's gestures. Further, the HMID 200 is able to sense the patient's auditory responses. In one example, when the probe 158 is at the Primary Motor Cortex, the HMD 200 may prompt the patient to follow a virtual object VO across the HMD display 208 and monitor eye movement to evaluate motor control. In another example, when the probe 158 is at the Auditory Nerve, the HMD 200 may prompt the patient through verbal instructions to identify sounds or melodies to evaluate hearing. Although one example of switching between senses is provided, it is contemplated that the HMD 200 and the surgical system 100 are capable of automatically changing the context of the testing and evaluation based on the location of the surgical tool 109 relative to critical brain regions CBR.

After the patient's baseline responses are recorded and the patient's brain is mapped, the procedure then turns to removing the target anatomy TA (e.g., tumor; diseased tissue). Turning to FIGS. 8A-8D, a resection and/or removal tool, such as powered forceps 160 and/or suction tool 156, may be used to remove the target anatomy TA. FIG. 8A illustrates an initial removal of the target anatomy TA with the suction tool 156. In this example, the suction tool 156 is within the boundary of the target anatomy TA, and the system 100 has established that the suction tool 156 is likely not going to interfere with critical brain area 256. During the removal of the target anatomy TA, the patient may be stimulated with prompts and/or given a number of commands to continue to verify that the critical brain area 256 outside of the virtual boundary VB are not negatively affected, such as described above during mapping. For example, the same or similar prompts and/or commands are communicated through HMD 200 to the patient to elicit responses corresponding with the critical brain areas which the surgical tool 109 may be close to. As with the brain mapping, monitoring functionality of the patient is paramount. During the removal of the target anatomy TA, stimulation by the surgical tool 109 to the patient may be conducted throughout the procedure to continuously monitor the patient's functionality. FIGS. 7B, 7C, and 7D show the suction tool 156 positioned close to virtual boundary VB, such that the suction tool 156 is approaching critical brain areas 250, 252, 254. To prevent removal of critical brain areas 250, 252, 254 during removal of the target anatomy TA, as the surgeon switches between removal and testing, the HMD 200 continues to prompt the patient in order to elicit responses based on the location of the surgical tool 109 relative to each critical brain areas 250, 252, 254, 256 of the critical brain regions CBR. For example, in FIG. 7B, when the suction tool 156 approaches critical brain area 254, and the surgeon switches to probe 158 to check functionality of the patient, the HMD 200 provides prompts to the patient to monitor the patient's sense of touch. Similarly, as seen in FIG. 7C, the suction tool 156 is seen approaching the critical brain area 250. The HMD 200 provides prompts and commands to the patient to elicit responses relating to cognitive ability. As the target anatomy TA is further removed, as shown in FIG. 7D, the suction tool 156 is shown approaching critical brain area 252. In this example, HMD 200 provides prompts and commands to elicit a response from the patient relating to cognitive functionality. In some examples, the prompts and/or commands relating to critical brain area 252 may be the same as the prompts and/or commands relating to critical brain area 250. In some examples, the prompts and/or commands relating to critical brain area 252 may be different from the prompts and/or commands relating to critical brain area 250. In some examples, when there is a reduction in functionality of the patient, the surgical tool 109 (e.g., suction tool 156) may have crossed the virtual boundary VB into one of the critical brain areas 250, 252, 254, 256 or is within a threshold distance to the virtual boundary VB that there is an observable difference in the patient's responses. As described above, the HMD 200 automatically changes the evaluation testing based on the location of the surgical tool 109 relative to critical brain regions CBR.

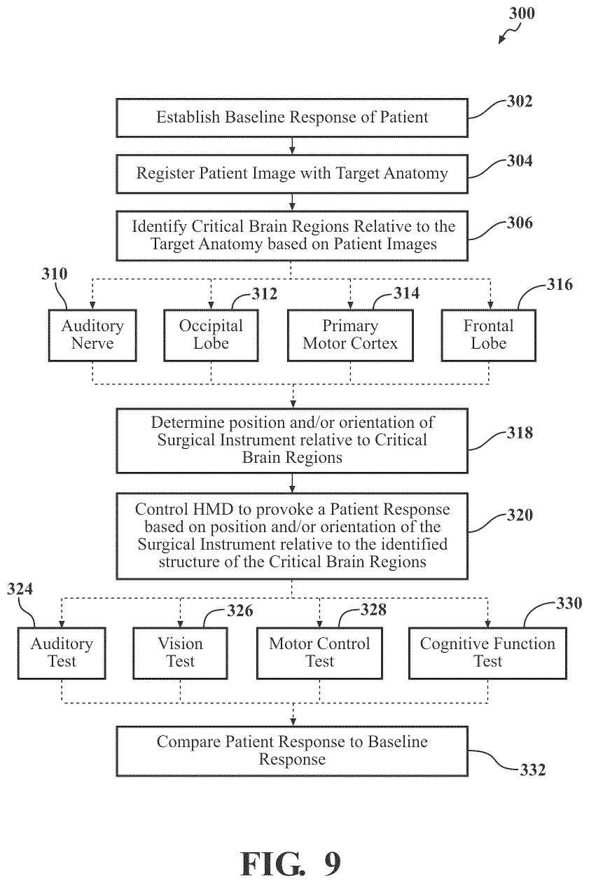

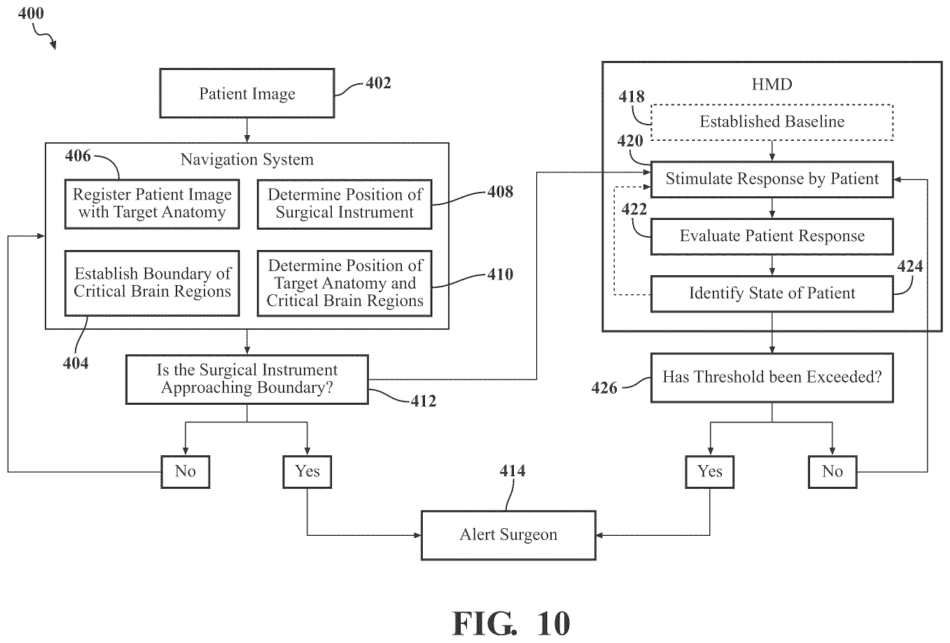

In view of the several techniques described above, FIGS. 9 and 10 each illustrate an example flowchart of an example method 300, 400 that may be performed to test and evaluate a patient for establishing and comparing anatomical and/or cognitive function. The steps shown in FIGS. 9 and 10 are provided for illustrative purposes to explain example methods for testing and evaluating a patient utilizing HMD 200 during a craniotomy. The steps shown in FIGS. 9 and 10 are not intended to limit the scope of any technique described herein. Any of the techniques described above may stand alone and provide advantages independent of the other techniques. More or less steps could be part of the methods 300, 400 in FIGS. 9 and 10. Additionally, steps could be implemented in an order different from what is shown and described. The technical details supporting each step have been presented above and are not repeated in detail for simplicity. The steps may be executed by any appropriate controllers/computing systems which may include but are not limited to, the camera controller 142, the navigation controller 110, the connectivity system CS, the computing system C, the tool controller, or the HMD controller 210.

Turning to FIG. 9, one example method 300 of a procedure utilizing HMD 200 during an awake craniotomy is illustrated as a flow chart. Starting with step 302, a baseline response for the patient is established. This may be performed with or without the HMD 200. During the procedure of step 302, a patient is asked a series of questions relating to problem solving, touch, hearing, sight, and other similar sensations and attributes to establish a baseline of brain activity and performance. This may be used to monitor the functionality of the patient during the craniotomy.

Next, step 304 includes registering the patient image (see FIGS. 5-6) with the navigation system 104. As described above, the navigation system 104 may use trackers 144, 146 to monitor and track the position of the patient which is registered with the patient images that display the target anatomy TA to be removed. For the purposes of this example, the target anatomy TA is a tumor for removal.

Step 306 may occur preoperatively and/or intraoperatively after a portion of the skull of the patient is removed to expose the brain. To identify the structures of the brain, the navigation system 104 registers the patient images with the surgical system 100. The surgical system 100 may be configured to identify critical structures of the brain, such as target anatomy TA, such as a tumor, and critical brain regions CBR illustrated in FIGS. 4A-4B, 5-6, 7A-7D, and 8A-8D. The surgical system 100 may automatically identify the target anatomy TA or may be guided by a medical professional. Some non-limiting example critical brain regions CBR that may be identified by the surgical system 100 are the auditory nerve 310, the occipital lobe 312, the primary motor cortex 314, the frontal lobe 316, or any other critical brain structure, such as other nerves, arteries, and/or tissues. See FIGS. 4A and 4B for annotated brain locations.

In some examples, such as shown at step 318, a surgical tool 109, such as a probe 158, may be tracked relative to the target anatomy TA (see FIGS. 6A-6D) and the critical brain structures CBR, such as critical brain areas 250, 252, 254, 256. The navigation system 104 tracks the position of surgical tool 109 (e.g., powered forceps 160, probe 158, suction tool 156) relative to the target anatomy TA and critical structures of the brain.

In step 320, the surgical tool 109 may be used with the HMD 200 to determine the effect of removal of the target anatomy TA in areas close to critical brain regions CBR, by stimulating particular regions until a notable reduction in ability is observed by the HMD 200. Some example critical brain regions CBR are the auditory nerve, occipital nerve, primary motor cortex, frontal lobe, and/or any similar areas of the brain which control sight, touch, smell, hearing, feeling, cognitive functions, and/or the like (see FIGS. 4A and 4B). Additional, non-limiting examples of the critical brain regions CBR are shown in FIGS. 4A and 4B. The HMD 200 may be configured to record the patient response in order to determine whether an area of the brain is affected by the stimulation. The navigation system 104 tracks the surgical tool 109 (such as the stimulation probe 158) relative to the target anatomy TA and the critical structures CBR, so that the HMD 200 may provide appropriate commands and/or prompts to the patient to elicit a response in order to record and analyze the patient response relative to the baseline.

The HMD 200 may be configured to automatically prompt and/or command through instructions and/or questions to the patient to respond to a virtual object VO or auditory command while the surgical tool 109 stimulates a portion of the brain. Depending on the area of the brain that the target anatomy TA is located relative to the critical brain regions CBR, one or more tests corresponding to one or more senses/cognitive functions may be provided to the patient through the HMD 200. For example, the registered images and the surgical tool 109 (e.g., probe 158) identify and/or verify critical brain regions CBR which the target anatomy TA is touching or adjacent to. Depending on the critical brain region CBR that the target anatomy TA is near, the HMD 200 may output testing commands to the patient to assist in determining a boundary of the critical brain region CBR relative to the target anatomy TA that is planned for removal without causing a reduction in patient functionality. The surgical system 100 may determine with the navigation system 104 and/or based on the patient responses whether the surgical tool 109 is approaching the virtual boundary VB of the critical brain regions CBR relative to the target anatomy TA being removed. For example, as the surgical tool 109 approaches the virtual boundary VB between the target anatomy TA and critical brain regions CBR, the surgical tool 109 may be turned off, an alert may prompt the surgical team, a monitored characteristic (e.g., by surgical team, HMD 200, or both) of the patient may change, or a combination thereof. In some other examples, the surgical tool 109 (such as probe 158) will be applied to an area of the brain and moved until there is an observed change in a particular function of the patient as compared with the baseline (described further below), such as a reduction in hearing or blurry vision. Testing the patient with the HMD 200 and probe 158 continues intermittently as the surgeon removes parts of the tumor (target anatomy TA) with resection tools (suction tool 156, powered forceps 160) to allow for surgeons to remove as much tumor as possible while preserving critical brain functions.

In one example, as shown in box 324, when the surgical tool 109 is near the auditory nerve, the HMD 200 will display virtual objects VO and/or produce verbal instructions to elicit a response by the patient to evaluate the hearing of the patient. Similarly, in another example, such as shown in box 326, when the surgical tool 109 is near the occipital lobe, the HMD 200 will display virtual objects VO and/or produce auditory instructions to elicit a response by the patient to evaluate the vision of the patient. In another example, such as shown in box 328, when the surgical tool 109 is near the primary motor cortex, the HMD 200 will display virtual objects VO and/or produce auditory instructions to elicit a response by the patient to evaluate the motor functions of the patient (e.g., move fingers, wiggle toes, describe sensation). In a further example, such as shown in box 330, when the surgical tool 109 is near the frontal lobe, the HMD 200 will display virtual objects VO and/or produce auditory instructions to elicit a response by the patient to evaluate the cognitive functions of the patient (e.g., simple math, answer questions, etc.).

As an example, in FIGS. 7A-7D and FIGS. 8A-8D, the surgical tool 109 (e.g., probe 158; suction tool 156) is shown moving around the target anatomy TA relative to critical brain regions CBR. As the surgical tool 109 is moved toward a particular critical brain region CBR, the HMD 200 may change the commands to the patient to prompt a response, such as described above. Since the target anatomy TA may be of a size which interferes with multiple critical brain regions CBR, the HMD 200 will change the testing scheme based on the position and/or orientation of the surgical tool 109 relative to the critical brain region CBR. In one non-limiting example, when the surgical tool 109 approaches the Broca's area of the brain, the HMD 200 monitors language of the patient by asking and/or commanding patients to complete tasks like counting, reading, and object naming. While using the surgical probe 158, direct electrical stimulation is applied to language areas, temporarily disrupting language-related abilities of the patient. The HMD 200 tests whether the patient is speaking and responding properly as compared with the baseline response, and if the stimulation of Broca's area of the brain interferes with the patient's ability to count, then that area is labeled as a critical brain region CBR and avoided when the tumor is removed. Alternatively, and/or additionally, if stimulation by the surgical tool 109 of a certain area (e.g., primary motor cortex, occipital lobe, frontal lobe) activates movement (or the lack thereof), reduction in cognition, and/or results in a sensation such as tingling, then that area is labeled as a critical brain region CBR and protected during the procedure.

As previously described with reference to establishing the baseline, the HMD 200 may record the patient responses with the sensing system 219 of the HMD 200. The HMD 200 may sense information or process sensed information, such as patient responses, with the HMD controller 210, the video camera 214, tracking sensors 212, IMU 216, and/or input sensors 217. In some examples, the patient response data is captured and processed partially or entirely on the HMD 200. In other examples, the patient response data may be processed by one or more controllers of the surgical system 100, such as the navigation controller 110, HMD controller 210, the remote sever RS, or combination thereof.

At step 332, the patient responses to the HMD 200 may be processed and analyzed to determine whether the response during stimulation by the surgical tool 109 is below a baseline response. To determine the quality of the patient response, the HMD 200 may record movements, answers, and/or comprehension based on the output of the HMD 200 to the patient to elicit the patient response. For example, during stimulation by the surgical tool 109, if the patient begins slurring speech, eye movement becomes erratic, or eye movement ceases, then the patient may be experiencing an adverse reaction to the stimulation by the surgical tool 109, indicating that the surgical tool 109 is affecting the critical brain regions CBR. In some examples, a response by a patient to stimulation by the surgical tool 109 may be gradual, such that the response may be 5 percent of the baseline response, 10 percent of the baseline response, 20 percent of the baseline response, or even 50 percent of the baseline response. In some examples, a healthcare professional may work with the patient preoperatively to determine an appropriate amount of function (e.g., reduced sight, cognition, speech, hearing, etc.), such that the threshold may reduce function of the patient in order to remove more of the target anatomy TA. Alternatively, the patient and the healthcare professional may work together to prioritize function of the patient over target anatomy TA removal. In each of these examples, the threshold may be varied depending on the patient's desired outcome.

If the difference between the baseline and the patient responses is greater than the predetermined threshold of functionality, the surgical system 100 may produce a response. The HMD 200 analyzes the patient response against an initial response to similar prompts administered while establishing the baseline. As described above, the HMD 200 uses the sensing system 219 to record, store, and analyze the patient responses. In some examples, when the response is below the predetermined threshold amount, the surgical system 100 may reduce power to the surgical tool 109, produce an alert, the like, or a combination thereof.

As seen in FIG. 10, flowchart 400 is illustrated. Starting at box 402, one or more patient images are taken/obtained and sent to the navigation system 104. The navigation system 104 facilitates registering the patient image with the target anatomy TA (e.g., tumor) at box 406. As described above, patient trackers 144, 146 may be used with the navigation system 104 to facilitate registration of the patient image(s) with the patient. The navigation system 104 also determines the position and/or orientation of the surgical instrument 109 (box 408) relative to the position of the target anatomy TA and critical brain regions CBR (box 410). As the procedure is performed, the boundary of the target anatomy TA is established relative to the critical brain regions CBR at box 404. The operation of the navigation system 104 may be constant throughout the procedure.

As the navigation system 104 is tracking the position and/or orientation of the surgical instrument 109 relative to the target anatomy TA, the navigation system 104 monitors the distance of the surgical instrument 109 from the established boundary(s) 404 of the target anatomy TA. If the surgical tool 109 is within a pre-determined distance of the boundary 412, the surgeon and/or medical staff are alerted (step 414). When the surgical system 100 monitors the position of the surgical tool 109 relative to the critical brain regions CBR, the HMD 200 monitors patient functionality by prompting the patient to respond to record their response for accuracy compared with the established baseline (described below with reference to 420, 422, 424). In some examples, in addition to an alert 414, the surgical instrument 109 may be turned off. Alternatively, when the surgical instrument 109 is not approaching the established boundary 412, no alert is sent, and the navigation system 104 continues to track the position and/or orientation of the surgical instrument 109 relative to the boundary of the target anatomy TA.

Turning to the right side of the chart 400, the HMD 200 is used to establish a baseline 418 of the patient's senses and cognitive function is established. As discussed above, the baseline 418 may be pre-operatively and/or intraoperatively established. As the surgical tool 109 is moved and actuated to provide a stimulus to the critical brain regions CBR, the HMD 200 is providing a stimulus (e.g., displaying virtual object VO and/or making sound) to which the patient is responding to (stimulate response by patient, step 420). The patient response at 420 is then evaluated at step 422 against the baseline 418 to determine the state of the patient 424. At step 426, the state of the patient is determined by comparing the intraoperative patient response to the baseline, and is dependent on whether the patient response is within a pre-determined threshold of the established baseline 418. Additionally, and/or alternatively, the patient responses may be assisted by machine learning and/or artificial intelligence to better understand and identify whether the patient is experiencing an adverse reaction to stimulation (e.g., electrical stimulation; removal of tissue), as compared with the baseline responses. For example, the HMD 200 may be configured to utilize machine learning and artificial intelligence to record, analyze, and monitor the patient with the suite of input sensors 217, such that more slight variations in patient functionality may be identified as a reduction in function based on the various sensor inputs that may assist the surgical team in identifying issues that may otherwise be missed by normal testing.

At 426, if the patient response is the same as the established baseline 418, the patient is in a normal state, and the process steps 420, 422, and 424 are repeated. Conversely, at 426, if the patient response is lower than the established baseline, the patient may be considered to be in a negative state, and an alert is provided to the surgeon and/or medical staff at 514.

The present teachings provide for a better and more precise patient data collection system by utilizing xR (extended reality) headsets on the patient. The xR headsets are configured to continuously monitor and process patient information pre-operatively, intraoperatively, and/or post-operatively. The xR headset worn by the patient connects with the surgical system and is configured to store and present the patient responses with the medical team.

Several implementations have been discussed in the foregoing description. However, the implementations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

What is claimed is:

1. A surgical system comprising:
a navigation system configured to track a pose of a surgical instrument relative to a pose of a target anatomy of a patient;
a head-mounted device (HMD) configured to be worn by the patient during surgery; and
a control system in communication with the navigation system and the HMD, the control system configured to:
register one or more images with the target anatomy;
identify a structure of the target anatomy based on the one or more images of the target anatomy;
detect the pose of the surgical instrument being proximate to the identified structure of target anatomy; and
based on detection of the pose of the surgical instrument being proximate to the identified structure, control the HMD to generate an output to provoke a response of the patient.

2. The surgical system of claim 1, wherein:
the target anatomy is a brain of the patient;
the identified structure is a specific brain region of the patient; and
the control system controls the HMD to generate the output in a manner that is specifically designed to provoke the response of the patient to enable a functional evaluation of the specific brain region.

3. The surgical system of claim 1, wherein:
the HMD comprises a display that is visible by the patient; and
the output to provoke the response of the patient is provided on the display.

4. The surgical system of claim 3, wherein the output provided on the display of the HMD comprises a visual test or visual instructions to provoke the response of the patient.

5. The surgical system of claim 1, wherein the HMD comprises a speaker system that is audible to the patient, and wherein the output to provoke the response of the patient is provided by the speaker system.

6. The surgical system of claim 5, wherein the output provided by the speaker system of the HMD comprises an audible test or audible instructions to provoke the response of the patient.

7. The surgical system of claim 1, wherein the HMD comprises one or more sensors configured to monitor the response of the patient.

8. The surgical system of claim 7, wherein the one or more sensors comprise a camera to monitor eye movement response of the patient.

9. The surgical system of claim 7, wherein the one or more sensors comprise a camera to monitor a blinking frequency response or a blinking closure time response from eyes of the patient.

10. The surgical system of claim 7, wherein the one or more sensors comprise a microphone to monitor a voice response from the patient.

11. The surgical system of claim 7, wherein the one or more sensors comprise a gesture sensor to monitor a gesture response from the patient.

12. The surgical system of claim 7, wherein, based on the one or more sensors, the control system is configured to evaluate the response of the patient.

13. The surgical system of claim 12, wherein, based on the one or more sensors, the control system is configured to evaluate whether the response of the patient is acceptable.

14. The surgical system of claim 13, wherein the control system is configured to utilize a machine learning model to evaluate whether the response of the patient is acceptable.

15. The surgical system of claim 13, further comprising a display device, and wherein the control system is configured to control the display device to present a notification or alert regarding whether the response of the patient is acceptable.

16. The surgical system of claim 13, wherein the control system is configured to:
detect interaction of the surgical instrument with the identified structure; and
continuously evaluate whether the response of the patient is acceptable during interaction of the surgical instrument with the identified structure.

17. The surgical system of claim 1, wherein the control system is configured to:
detect a condition during surgery; and
control the HMD to change the output based on the detected condition.

18. The surgical system of claim 17, wherein the detected condition comprises use of a second surgical instrument, instead of and different from the surgical instrument, proximate to the identified structure.

19. The surgical system of claim 1, wherein:
the control system is configured to establish a virtual boundary based on the identified structure of the target anatomy;
the navigation system is configured to determine the pose of the surgical instrument relative to the virtual boundary; and
the control system is configured to control the output of the HMD based on the pose of the instrument relative to virtual boundary.

20. A head-mounted device (HMD) for use with a navigation in awake brain surgery of a patient, the HMD comprising:
a support structure to be worn by the patient during surgery;
a display supported by the support structure and being visible by the patient;
a speaker system supported by the support structure and being audible to the patient;
one or more sensors supported by the support structure; and
an HMD controller coupled to the display, speaker system, and one or more sensors, wherein the HMD controller is configured to:
obtain an instruction from a navigation system in response to the navigation system detecting that a surgical instrument is proximate to a specific brain region of the patient;
based on receipt of the instruction, control one or both of the display and the speaker system to generate an output to provoke a response of the patient; and
utilize the one or more sensors to monitor the response of the patient and functionally evaluate the specific brain region.
